(19) United States
(12) Patent Application Publication
(10) Pub. No.: US 2010/0144005 A1
(43) Pub. Date: Jun. 10, 2010

(54) PROCESS FOR ISOLATING MICROORGANISMS FROM SAMPLES AND SYSTEM, APPARATUS AND COMPOSITIONS THEREFOR

(76) Inventors: Cesar G. I. Bin Kingombe, Sarsfield (CA); Anthony N. Sharpe, Almonte (CA)

Correspondence Address: NATIONAL RESEARCH COUNCIL OF CANADA, 1200 Montreal Road, Building M-58 Room EG-12, OTTAWA, ONTARIO, K1A 0R6, CA

(21) Appl. No.: 12/451,084

(22) PCT Filed: Apr. 18, 2008
(86) PCT No.: PCT/CA2008/000811

§ 371 Date: Oct. 26, 2009

Related U.S. Application Data
(60) Provisional application No. 60/924,001, filed on Apr. 26, 2007.

(51) Int. Cl. C12N 13/00; C12M 1/42

(52) U.S. Cl. 435/173.9; 435/308.1

(57) ABSTRACT

A process for isolating microorganisms from samples, particularly Shigella spp. from food samples, and a system, apparatus and composition therefor are provided. Magnetic particles are used to capture microorganisms and a system having separate magnetically-based apparatuses for collecting, concentrating and retrieving is used to isolate the magnetic particles having bound microorganisms. The apparatus for concentrating magnetic particles utilizes a small magnet assisted by vibration to concentrate collected particles at a localized region on the bottom of a container. The process, system and apparatus of the present invention are simple and inexpensive providing improved magnetic particle recovery adaptable to large scales.

### CROSS-REFERENCE TO RELATED APPLICATIONS

This application claims the benefit of U.S. provisional patent application U.S. Ser. No. 60/924,001 filed Apr. 26, 2007, the entire contents of which is herein incorporated by reference.

### FIELD OF THE INVENTION

The present invention relates to a process for isolating microorganisms from samples, particularly food samples, and a system, apparatus and composition therefor.

### BACKGROUND OF THE INVENTION

The use of magnetic particle technology, particularly antibody-coated magnetic beads (immunomagnetic beads), for the selective isolation of microorganisms in microbiology in general and in food and environmental microbiology in particular is becoming more widely used. Different systems and individual pieces of equipment have been developed to assist in the use of magnetic particles.

Many systems have been developed for collecting magnetic beads from small scale volume samples. Such systems typically handle samples of volumes from 1 ml (Eppendorf tubes, e.g. MagneSphere Technology Magnetic Separation Stand, Promega Cat. # Z5331, Z5332, Z5333 (two-position), Z5341, Z5342 and Z5343 (twelve-position) up to about 50 ml (Falcon tubes, e.g. PolyAtract System 1000 Magnetic Separation Stand, Promega Cat. # Z5410). Magnets are used to concentrate the magnetic beads at the side of the tubes and a pipette is used to either remove supernatant liquid or remove the beads directly. Magnetic pipette's, for example the PickPen™ product, may be used to remove the magnetic beads directly. Such systems are not well adapted for large sample sizes and large volumes of medium, thereby limiting their usefulness in the isolation of some types of microorganisms, e.g. Shigella spp according to Health Canada, Compendium of Analytical Methods for Food and Drug Administration, Bacteriologic Analytical Manual.

Automated systems, for example the Pathatrix™ system from Matrix MicroSciences, peristaltic pumps, tubes and in-line filters to minimize human handling of samples. Such systems are very expensive, have problems with bead loss on the filters due to the formation of bio-films, and are prone to spillage when transferring the beads from the system.

Other systems, for example the Kingfisher™ system, are based on the use of electromagnetic pins for capturing magnetic beads from an array of small-sized tubes (<2 ml) and transferring the beads to new tubes for further processing. The electromagnetic pins may be used to hold beads while exchanging tubes, and then to release the beads into the new tubes. Their applications are limited to purification of DNA from PCR products or from gels.

Various other systems use magnets in various ways to process magnetic beads. For example, United States Patent Publication 2005/0013741 published Jan. 20, 2005 discloses a device for immobilizing and re-suspending magnetic particles during washing and elution steps. The device comprises two permanent magnets which are movable along the side of a tube containing the magnetic particles in a liquid.

Shigella spp. presents a greater challenge to its rapid detection as these microorganisms generally contaminate food samples at much lower concentrations than other species of microorganisms and are poor competitors. We believe the detection and/or isolation of Shigella spp., especially from food samples, requires processing samples on a larger scale, preferably assisted with specific antibodies coated on magnetic beads.

There remains a need for simple, inexpensive and flexible processes, systems and apparatuses for selectively isolating microorganisms from samples, especially food samples, particularly rapidly and on a large scale, and particularly for Shigella spp.

### SUMMARY OF THE INVENTION

There is provided a process for selectively isolating microorganisms from a sample, the process comprising: providing a container containing a medium for selectively enriching the microorganisms; adding the sample containing the microorganisms to the medium; adding to the medium magnetic particles adapted to bind the microorganisms of interest; magnetically collecting the particles with bound microorganisms at a bottom of the container; aided by vibrating the container, magnetically concentrating the particles with bound microorganisms at a localized region on the bottom of the container; and retrieving the particles from the localized region with a magnetically assisted pipette.

There is further provided a system for recovering magnetic particles from a mixture in a container, the system comprising: a magnetic particle collector having a first magnet for attracting the magnetic particles to a bottom of the container; a magnetic particle concentrator having a second magnet for concentrating the magnetic particles collected on the bottom into a localized region on the bottom, the concentrator having means for vibrating the container to assist in movement of the magnetic particles to the localized region; and, a magnetic particle pipette having a third magnet for retrieving the magnetic particles concentrated in the localized region.

There is further provided an apparatus for concentrating magnetic particles into a localized region on a bottom of a container, the apparatus comprising: a support structure for supporting the container; a magnet positioned below the container when the container is supported on the support structure, the magnet having a magnetic field localizable at the region on the bottom of the container to concentrate the magnetic particles into the region, the magnetic field removable from the region to permit retrieval of the magnetic particles without interference from the magnet; and, means for vibrating the container to assist in movement of the magnetic particles to the localized region.

There is further provided a composition for selectively enriching Shigella spp. comprising: water, tryptone, potassium phosphate dibasic, potassium phosphate tribasic, sodium chloride, glucose, polyethyleneglycol sorbitan monooleate, and Crystal Violet.

The process, system and apparatus of the present invention are particularly useful for determining the presence of and/or assaying the amount of food pathogens in a food sample. Food pathogens include, for example, bacteria, parasites and viruses. Microorganisms are of particular interest, for example, bacteria. Microorganisms include, for example, Aeromonas spp., E. coli, Shigella spp., Salmonella spp., Listeria spp., Campylobacter spp., Clostridium spp., Vibrio spp., Staphylococcus aureus, and Yersinia enterocolitica. The process, system, apparatus and composition of the present invention are particularly useful for the isolation, detection, measurement and/or enrichment of Shigella spp. Shigella spp. includes, for example, S. boydii, S. dysenteriae, S. flexneri and S. sonnei.

The process, system and apparatus are particularly useful on a relatively large scale. Most prior art processes, systems and apparatuses are adapted for tubes having volumes on the order of a few milliliters, e.g. up to about 50 ml, and to small sample sizes. Small volume and sample size limits the efficiency and effectiveness of microorganism growth, detection and measurement, reduces capturing yield of the microorganism and contributes to the difficulty of handling samples and the possibility of false negative results. In Canada, (Health Canada, Compendium of Analytical Methods) and the USA (Food and Drug Administration, Bacteriologic Analytical Manual), the legal sample size is 25 g and the volume of enrichment broth is 225 ml. Further, tubes typically have rounded bottoms and are therefore more difficult to handle. In contrast, the present invention can be practiced at much larger scales with containers having flat bottoms. Containers having volumes of over 100 ml, or even over 250 ml, for example about 500 ml, can be easily handled. Use of flat-bottomed containers, for example Erlenmeyer flasks, beakers and evaporating dishes are preferred. Samples having masses over 10 grams can be used, for example 25 grams.

The process of the present invention involves providing a container containing a medium (e.g. an enrichment broth) for selectively enriching microorganism of interest and adding a sample containing the microorganism to the medium. The sample is preferably a food sample and the sample is preferably mixed with the medium initially before incubation. Microorganisms are then allowed to incubate for a time of from about 6 to about 24 hours. The microorganisms of interest will selectively grow on the medium, out-competing other microorganisms which may have been present in the sample. The medium is preferably a liquid (e.g. a nutrient broth). Media for enriching microorganisms are commonly known in the art, for example as disclosed in Health Canada, Compendium of Analytical Methods or Food and Drug Administration, Bacteriologic Analytical Manual. A liquid medium is preferred since a solid medium will need to be mobilized in a liquid prior to adding magnetic particles.

For enriching Shigella spp., Shigella broth and a broth containing the composition of the present invention (known as Shiga broth) are preferred. The composition of the present invention advantageously contains Crystal Violet which suppresses the growth of Bacillus, common interfering bacteria in Shigella assays which are not eliminated on currently available media used to selectively grow Shigella spp., e.g. Shigella broth. The composition of the present invention may also include other antibiotics, for example novobiocin.

Before adding the magnetic particles to the medium, it is sometimes desirable to filter the enriched medium. It is particularly desirable when a very evident bio-film has formed. When filtering is desired, the filter preferably does not inhibit the microorganisms of interest. Any suitable filter may be used. In one embodiment, a filter assembly may be used to filter the entire medium, the filter assembly comprising a Millipore Express Plus 0.22 μm filter in which the 0.22 μm filter is removed and replaced with a foam pad from Filtaflex Ltd. that does not inhibit microorganisms.

The magnetic particles added to the medium are adapted to bind the microorganisms of interest, preferably by means of specific antibodies conjugated to the magnetic particles. Preferably, the magnetic particles are added in an amount of about 50-100 μl per 250 ml of medium, for example about 100 μl per 250 ml. Preferably, the magnetic particles are added to the medium with mixing and then the medium incubated to allow the microorganisms to bind to the magnetic particles. Incubation time is preferably about 15-30 minutes.

The magnetic particles may be spherical or non-spherical. Spherical particles are preferred as non-spherical particles may kill microorganisms. Some examples of magnetic particles include Cortex Megacell™-Streptavidin magnetic particles, Cortex Megabeads™-Streptavidin CM3454 (8.8 μm particle size and coated with magnetizable polystyrene/iron oxide particles), Cortex Megabeads™-Streptavidin CTM-CM019 (15.6 μm particle size and coated with polystyrene copolymer/iron oxide particles), Dynabeads™ M-280-Streptavidin (3-4 μm particle size), and Genpoint BugTrap™ magnetic beads.

Cortex Megabeads™-Streptavidin CTM-CM019 (15.6 μm particle size and coated with polystyrene copolymer/iron oxide particles) conjugated to Shigella antibodies (monoclonal and/or polyclonal) and Genpoint BugTrap™ magnetic beads which are universal for capturing gram positive and negative bacteria, have diameters in a range of about 15 μm. These are preferred over the non-spherical Cortex Megacell™-Streptavidin magnetic particles. More preferable yet are the BugTrap™ binding beads from Genpoint AS, Oslo, Norway, which have diameters in a range of about 2.5-15 μm. The Genpoint BugTrap™ binding beads can be used even when a bio-film is present in the medium, and these beads are in a ready to use kit and are coated with a ligand for capturing Gram positive as well as Gram negative pathogenic bacteria (Canadian Patent Publication 2,397,067 published Jul. 26, 2001).

Preferably, the particles are immunomagnetic particles, more preferably immunomagnetic beads, comprising one or more monoclonal and/or polyclonal antibodies that specifically bind to an antigen on the microorganisms of interest. A mixture of immunomagnetic particles comprising different antibodies specific for different species of the microorganism genus of interest may be used. Some examples of species specific antibodies for Shigella spp. are: monoclonal anti-Shigella sonnei; clone 1028/437 cat. # MAB755 from CHEMICON-Millipore; Polyvalent D from Danka Seiken Co. for S. sonnei; anti-Shigella IgG with biotin from Cortex Biochem for S. boydii, S. flexneri, and S. dysenteriae (cat. # CR1243RB); and, polyclonal antibody to Shigella spp. with biotin from Acris Antibodies GmbH (S. boydii ATCC #8700, S. flexneri ATCC #29903 and S. dysenteriae ATCC #13313). Antibodies to other specific species may be raised by known methods and incorporated into an immunomagnetic particle.

Immunomagnetic particles typically comprise a core magnetic particle coated with an avidin (e.g. streptavidin), in turn coated with biotin. The biotin is in turn coated with the antibody or antibodies. Methods for constructing immunomagnetic particles are generally known in the art (e.g. Safarik, I. and Safarikova, M. “Magnetic techniques for the isolation and purification of proteins and peptides.” BioMagn. Res. Technol. 2 (2004) 7).

The magnetic particles with bound microorganisms are then recovered by magnetically collecting the particles at a bottom of the container, magnetically concentrating the particles at a localized region on the bottom of the container, and retrieving the particles from the localized region with a magnetically assisted pipette. To accomplish this, the system and apparatus of the present invention are preferably employed.

The system comprises a magnetic particle collector, a magnetic particle concentrator and a magnetic particle pipette that cooperate to recover the magnetic particles from the medium.

The magnetic particle collector is used first and comprises a magnet that attracts the magnetic particles to the bottom of the container that contains the medium. The magnet is preferably large, preferably having a surface at least as large as half the area of the bottom of the container. The magnet may be at least as large as the bottom of the container or larger than the bottom of the container. The magnet preferably has a large and extensive magnetic field to attract magnetic particles as far away as the upper surface of the medium in the container. Thus, magnetic particles throughout the entire medium are attracted to the bottom of the container. The magnet may be any suitable shape, for example cylindrical. Thickness of the magnet is preferably at least one-fifth that of its diameter. The magnet may be, for example, a permanent magnet or an electromagnet. A block magnet such as the Huge-Field Magnet from Filtaflex Ltd. is one embodiment of a suitable magnet. The magnet may be made of any suitable material, for example, neodymium-iron-boron alloy or samarium-cobalt alloy. The magnetic particle collector may have a protective cover for storing it when not in use. An enclosure around the magnetic particle collector may be used to protect it from magnetic objects drawn to it from the surroundings. A label warning users about possible injury if magnetic objects are brought too near the magnet may also be affixed to the magnetic particle collector.

The magnetic particle concentrator is then used to concentrate the magnetic particles into a localized region on the bottom of the container. A preferred embodiment of the concentrator is an apparatus of the present invention.

To effect movement of the particles into the localized region, the apparatus comprises a magnet that is positioned below the container when the container is in the apparatus. The magnet has a magnetic field localizable at the region on the bottom of the container. The magnet may be a permanent magnet or an electromagnet. The magnet is preferably small having a surface significantly smaller than the bottom of the container, with the size of the magnet determining the size of the localized region. The magnet preferably has a magnetic field large enough to attract particles from the furthest edge of the bottom of the container. The localized region is preferably at the center of the bottom of the container and the magnet is preferably positioned under the center of the bottom of the container.

The magnetic field generated by the magnet is removable from the region once the magnetic particles have been concentrated there in order to permit retrieval of the magnetic particles without interference from the magnet. The magnet may be physically removed from the region to lessen or eliminate the effect of the magnetic field at the region. The magnet may be moved, for example, by use of a handle conveniently located on the concentrator, for example on a side or front. If the magnet is an electromagnet, the magnetic field may be removed by switching the electromagnet off.

To further assist movement of the particles to the localized region, the apparatus advantageously further comprises means for vibrating the container. Any suitable means for vibrating the container may be used, for example vibrating arm or arms, vibrating base, sonicator, etc. If a sonicator is used, care should be taken not to kill the microorganism. Vibration raises the particles slightly off the bottom thereby making it easier for the magnet to move the magnetic particles through the medium to the localized region without re-suspending the particles in the medium as a whole. Preferably, lateral vibrations are applied to the container. Vibrations should not be severe enough to shake the container thereby re-suspending the particles throughout the medium as a whole.

The apparatus further comprises a support structure for supporting the container. The support structure may comprise a base on which the container sits. The support structure may be composed of any suitable material, for example plastic (e.g. polycarbonate), metal (e.g. aluminum) or a combination thereof. To assist in determining whether the magnetic particles have all been concentrated into the localized region, all or part of the support structure, particularly the base, may be transparent. Further, the apparatus may further comprise one or more mirrors to assist in observing the bottom of the container from underneath the container. Furthermore, the apparatus preferably further comprises controls and gauges for controlling and displaying various operational parameters such as vibration time and speed.

The magnetic particle pipette is then used to retrieve the magnetic particles from the localized region. In one embodiment, the magnetic particle pipette is similar to commonly available micropipettes (e.g. Eppendorf, Gilson, PickPen™) with some differences. The magnetic particle pipette useful for the present invention is larger than either the Eppendorf, Gilson or PickPen™. Also, unlike the Eppendorf, the magnetic particle pipette is also equipped with a magnet to retain magnetic particles. Unlike the PickPen™, the magnetic particle pipette has a central plunger for particle retrieval and a side lever for tip removal, which reduces accidental loss of particles due to inadvertent activation of the side lever. The magnet may be a permanent magnet or an electromagnet.

After recovering the magnetic particles with bound microorganisms, the magnetic particles may be washed. Washing is preferably accomplished with TALON™ binding and washing buffer in a small volume container (e.g. an Eppendorf tube). Preferably, a buffer having a pH in a range of from about 7.5 to about 8.0 is used to wash the particles. The wash solution may be removed, for example with a pipette, after collecting the particles using the magnetic particle collector of the present invention or any other magnetic particle separation technology (e.g. MagneSphere Technology Magnetic Separation Stand, Promega Cat. # Z5331, Z5332, Z5333 (two-position). The magnetic particles with bound microorganisms may be assayed directly or frozen for storage at −80° C. until later analysis for downstream needs, e.g. isolation, serology, ELISA, DNA extraction, PCR, hybridization, etc. Freezing may be accomplished using, for example, a CryoStor™ (Innovatek Medical Inc., Vancouver, British Columbia, Canada).

Any suitable analytical technique may be used to detect and/or measure the microorganisms of interest that have been bound to the particles. For example: the particles may be plated on a medium (e.g. agar) and the microorganisms cultured; DNA may be extracted from the microorganisms on the particles and amplified with PCR; serology may be performed directly from the beads (e.g. add Shigella specific antibody and observe clumping); or an assay (e.g. ELISA) may be performed directly from the beads. Such techniques are well known in the art.

The collection, concentration and retrieval of magnetic particles in the process of the present invention have been divided into separate steps using separate apparatuses in a system. As a result, the present invention has a number of advantages. For example, in comparison to prior art, microorganisms may be isolated on a much larger scale, there are fewer problems with contamination, microorganism capture is more efficient and most or all of the magnetic particles may be retrieved, leading to more consistent and reproducible results. Since, the apparatuses used in the present invention are much less complicated, lower in cost and more amenable to scale-up than equipment required in many prior art processes, the entire process of the present invention is lower in cost than prior art processes. Further, the invention is particularly adaptable for effectively isolating Shigella spp., which are usually present in samples only at very low levels and heretofore have been difficult to isolate.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 is a side schematic view of a magnetic particle collector for collecting immunomagnetic beads on a bottom of an Erlenmeyer flask resting on the collector;

FIG. 2A is a schematic front perspective view of a magnetic particle concentrator for concentrating immunomagnetic particles into a localized region on a bottom of an Erlenmeyer flask resting in the concentrator;

FIG. 2B is a schematic rear perspective view of the magnetic particle concentrator depicted in FIG. 2A;

FIG. 2C is a schematic top perspective view of the magnetic particle concentrator depicted in FIG. 2A;

FIG. 2D is a schematic bottom perspective view of the magnetic particle concentrator depicted in FIG. 2A;

FIG. 3 is a front schematic view of a magnetic particle pipette for retrieving immunomagnetic beads from a localized region at a bottom of an Erlenmeyer flask;

FIG. 4A is a schematic front perspective view of a second embodiment of a magnetic particle concentrator of the present invention;

FIG. 4B is a schematic rear perspective view of the magnetic particle concentrator depicted in FIG. 4A;

FIG. 4C is a schematic top perspective view of the magnetic particle concentrator depicted in FIG. 4A;

FIG. 4D is a schematic bottom perspective view of the magnetic particle concentrator depicted in FIG. 4A;

FIG. 4E is a schematic side perspective view of the magnetic particle concentrator depicted in FIG. 4A;

FIG. 5A is a side schematic view of a second embodiment of a magnetic particle collector;

FIG. 5B is a side schematic view of a third embodiment of a magnetic particle collector;

FIG. 6A is a schematic plan view of a tilting frame of a third embodiment of a magnetic particle concentrator of the present invention;

FIG. 6B is a schematic view of a section along A-A of the tilting frame of FIG. 6A;

FIG. 6C is a schematic plan view of a chassis of the third embodiment of the magnetic particle concentrator;

FIG. 6D is a section view along C-C of the chassis of FIG. 6C together with an electric motor;

FIG. 6E is schematic side view of a vibrating assembly of the third embodiment of the magnetic particle concentrator;

FIG. 6F is a schematic plan view of the chassis together with the vibrating assembly of the third embodiment of the magnetic particle concentrator; and,

FIGS. 7A-7C depict schematic cross-section views of a second embodiment of a magnetic particle pipette.

### DESCRIPTION OF PREFERRED EMBODIMENTS

### Process, System, Apparatus and Composition

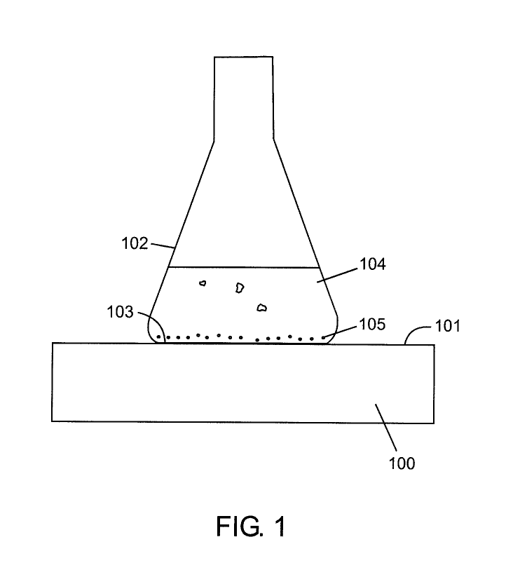

Referring to FIGS. 1-3, a first embodiment of a process of the present invention utilizing a system, apparatus and composition of the present invention is now described.

A nutrient broth of the present invention specific to Shigella spp. is prepared from a basal medium and an antibiotic supplement. The ingredients and their amounts are shown in Table 1.

TABLE 1
Basal Medium (pH = 7.0 ± 0.2)

| | | |
|---|---|---|
| Tryptone | 20.0 | g |
| Potassium phosphate dibasic (K₂HPO₄) | 2.0 | g |
| Potassium phosphate monobasic (KH₂PO₄) | 2.0 | g |
| Sodium chloride (NaCl) | 5.0 | g |
| Glucose | 1.0 | g |
| Tween ™ 80 (polyethyleneglycol sorbitan monooleate) | 1.5 | ml |
| Distilled water | 1.0 | L |

Supplement 1

| | | |
|---|---|---|
| Novobiocin | 50 | mg |
| Distilled water | 1.0 | L |

Supplement 2

| | | |
|---|---|---|
| Crystal Violet | 1 | g |
| Distilled water | 1.0 | L |

The basal medium (i.e. Shigella Broth by Difco™) is prepared by mixing the ingredient and heating the mixture to completely dissolve the various ingredients in the distilled water. The basal medium mixture is autoclaved at 121° C. for 15 minutes, and allowed to cool to 50° C. Supplement 1 is a 0.005% novobiocin solution sterilized by filtration through a 0.45 μm membrane filter. Supplement 2 is a 0.1% Crystal Violet solution prepared by suspending Crystal Violet in sterile distilled water. The two supplements are prepared separately. To prepare the final broth, (“Shiga” broth), 2.5 ml of supplement 1 and 1 ml of supplement 2 are aseptically added to 225 ml of the basal medium in a 500 ml Erlenmeyer flask and mixed thoroughly.

Immunomagnetic beads are prepared based on magnetic beads coated with streptavidin (e.g. Cortex Megabeads™-Streptavidin CTM-CM019, Dynabeads™ M-280-Streptavidin) or other ligands (e.g. Genpoint BugTrap™ magnetic beads). The coated beads are then conjugated with biotin coated anti-Shigella IgG (e.g. Polyvalent D, a S. sonnei specific antibody from Danka Seiken Co., Japan; and/or, anti-Shigella-Biotin IgG from Cortex Biochem, USA, which are specific to S. boydii, S. flexneri and S. dysenteriae; and/or Acris Antibodies GmbH (S. boydii ATC #8700, S. flexneri ATC #29903, and S. dysenteriae ATC #13313). A mixture of the various types of antibody-conjugated beads may be used in the process.

A food sample (25 g) suspected of being contaminated with Shigella spp. is added to 225 ml of the broth in a Stomacher bag and incubated for 10 min with gentle massage. The homogenate is then transferred into a 500 ml Erlenmeyer flask and incubated at 42° C. for 6 to 24 hours. After incubation, 50 to 100 μl of the mixture of immunomagnetic beads is added and mixed into the broth and the broth and beads mixture is incubated for 30 minutes to allow the Shigella bacteria to bind to the immunomagnetic beads.

Referring specifically to FIG. 1, immunomagnetic beads to which Shigella bacteria are bound are then collected on the bottom of the Erlenmeyer flask. Erlenmeyer flask 102 rests on magnetic particle collector 100, which is a large block magnet having upper surface 101 that is larger in surface area than the surface area of bottom 103 of the Erlenmeyer flask. The magnet is a Huge-Field Magnet from FILTAFLEX Ltd. Immunomagnetic beads 105 (only one indicated in the Figure) in broth 104 are drawn down to the bottom of the flask under the influence of the magnetic field of the magnet.

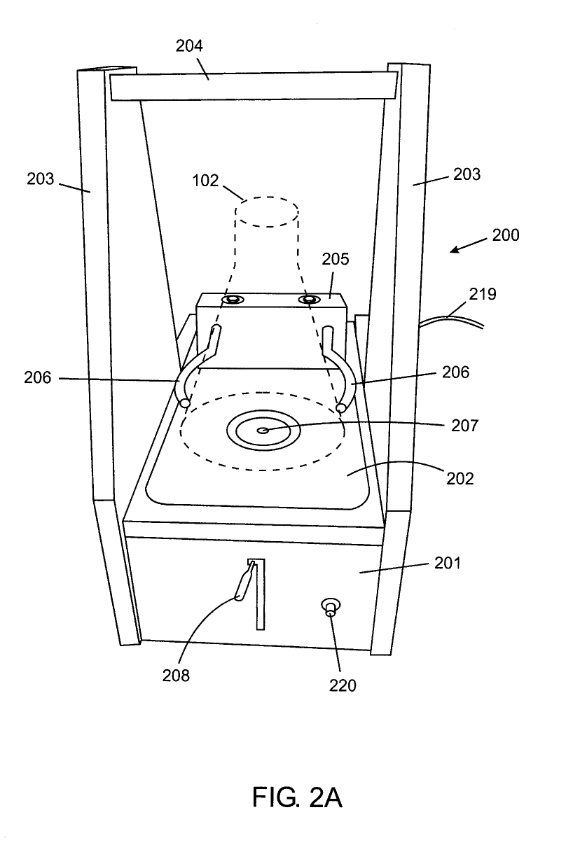

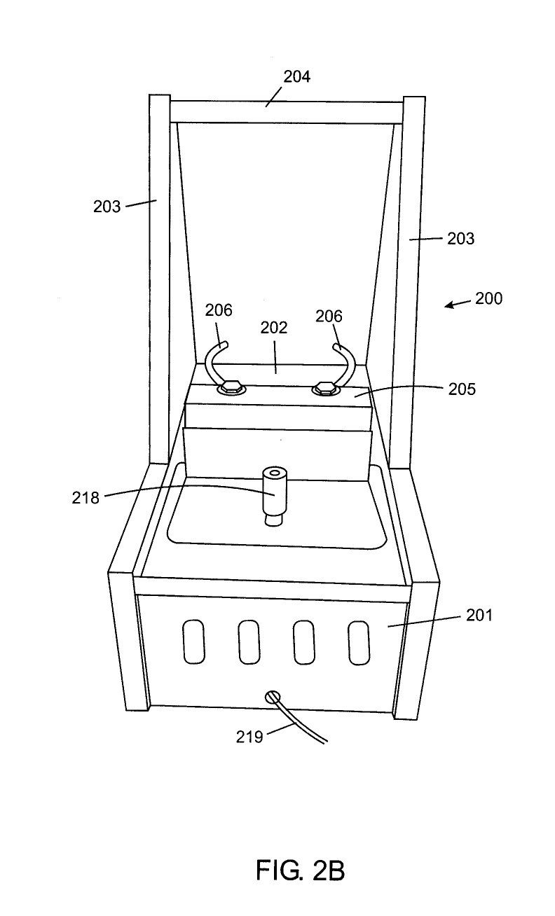

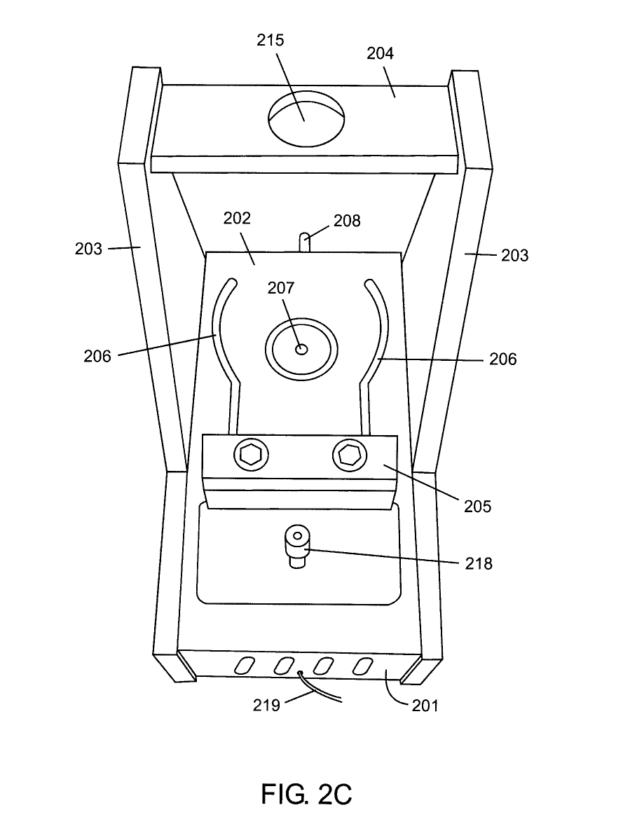

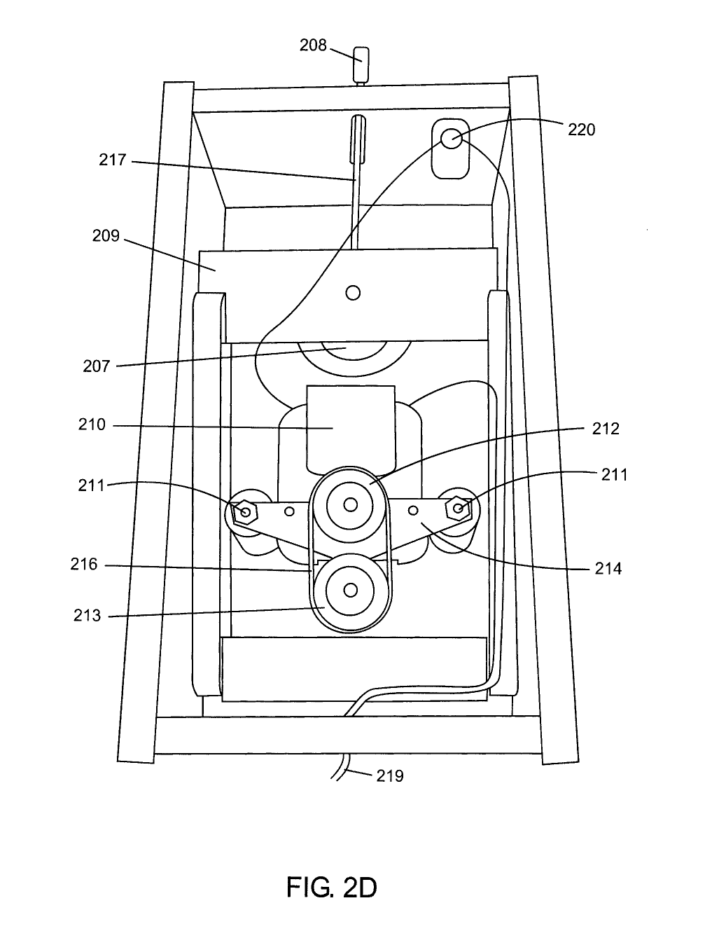

Referring specifically to FIGS. 2A-2D, after all of the immunomagnetic beads have collected on the bottom of the Erlenmeyer flask, Erlenmeyer flask 102 is placed in magnetic particle concentrator 200. Magnetic particle concentrator 200 comprises a support structure for supporting the flask, a magnet positioned below the flask for concentrating the immunomagnetic beads into the center of the bottom of the flask, and means for vibrating the flask.

The support structure comprises base 202 upon which the flask rests, housing 201 for housing components of the concentrator and a pair of side walls 203 for supporting top strut 204. Top strut 204 has aperture 215 through which a pipette may be inserted as will be described later. The support structure also comprises arm support 205 for supporting arms 206 which are part of the means for vibrating the flask.

Movable magnet 207 is mounted on magnet armature 209 and is movable up and down by actuation of handle 208 which moves lever 217 connected to the armature. The handle is connected to the lever. Magnet 207 is positioned just below the center of the bottom of flask 102. Depressing the handle moves the magnet down away from the flask. Raising the handle returns the magnet to its position just below the flask.

The means for vibrating the flask comprises a pair of arms 206 between which the flask rests. The arms are curved to accommodate the contours of the flask. The arms are connected to spindles 211 and the spindles connected to rocker bar 214. The rocker bar is mounted on first pulley 212 and the first pulley is mounted on a drive shaft of electric motor 210. Second pulley 213 is mounted on the housing and connected to the first pulley by belt 216. Counterweight 218 is connected to the second pulley by a shaft. Electricity is supplied to the motor from power cord 219 and the motor is switched on and off by actuation of switch 220.

To concentrate the immunomagnetic beads at the center of the bottom of flask 102 (shown in phantom in FIG. 2A), the flask is placed in the concentrator between arms 206 such that the arms are in contact with the flask, and magnet 207 is raised to its position just below the flask. Motor 210 is switched on and the motor rotates the drive shaft which in turn rotates first pulley 212. Rotation of the first pulley in turn rotates second pulley 213 by virtue of belt 216. Rotation of the second pulley is eccentric due to counterweight 218. The eccentricity of rotation of the second pulley creates a vibration in the first pulley which is transmitted through rocker bar 214 and spindles 211 to arms 206. Vibration of arms 206 is transmitted to flask 102 and thence to the flask's contents. Magnet 207 attracts the beads toward the center of the bottom of the flask, and vibration of the flask causes the beads to be slightly raised off the bottom of the flask thereby assisting in movement of the beads by eliminating friction between the beads and the bottom of the flask.

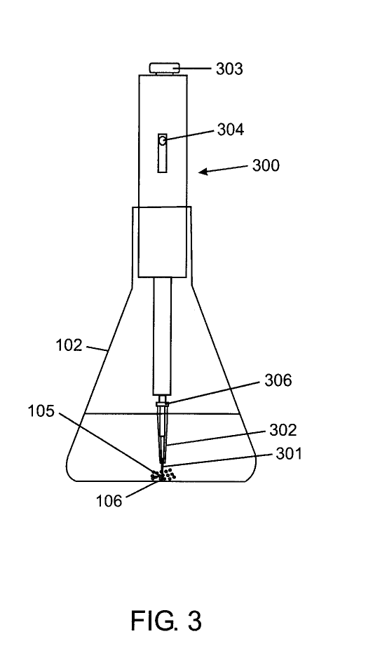

Referring specifically to FIG. 3 with further reference to FIGS. 2A-D, immunomagnetic beads 105 (only one indicated in the Figure) concentrated into center 106 of the bottom of Erlenmeyer flask 102 are then retrieved with magnetic particle pipette 300, without removing the flask from the concentrator. For clarity, the concentrator is not depicted in FIG. 3 and reference to the concentrator is in connection with FIGS. 2A-2D. Movable magnet 207 of concentrator 200 is lowered away from the bottom of the flask by actuation of handle 208 to reduce interference with the retrieval process. The pipette comprises extendable magnetic tip 301 which can be extended and retracted by action of spring-loaded plunger 303. The pipette also comprises removable rubber tip 302 which may be removed by action of spring-loaded slide 304.

The pipette operates as follows. A sterile rubber tip is first fitted over the magnetic tip by inserting the magnetic tip into the rubber tip up to stop 306. The rubber tip is held in place by friction. Magnetic tip 301 is extended by depressing plunger 303; and the pipette is inserted through aperture 215 in top strut 204 of the concentrator and thence into the flask so that the magnetic tip is in the concentrated beads. Immunomagnetic beads 105 collect on the magnetic tip. Keeping spring-loaded plunger 303 depressed, the pipette is withdrawn from the flask with the beads remaining attached to the magnetic tip. The magnetic tip is inserted into a wash solution and the beads transferred to the wash solution by withdrawing the magnetic tip allowing the beads to be pushed off the magnetic tip by the rubber tip. Withdrawing the magnetic tip is accomplished by releasing spring-loaded plunger 303. Rubber tip 302 is disposed of by depressing spring-loaded slide 304 which pushes the rubber tip off the end of the pipette.

Washing the recovered beads is accomplished with a washing and binding buffer solution (e.g. TALON™ binding and washing buffer from Dynal Biotech; B&W Buffer from the BugTrap Bacteria Isolation Kit from Genpoint AS, Oslo) having a pH of 7.5 to 8. The wash solution is removed, for example with a pipette, after collecting the beads using the magnetic particle collector of the present invention. The magnetic particles with bound microorganisms may be assayed directly or frozen for later analysis.

### Second Embodiment of Magnetic Particle Concentrator

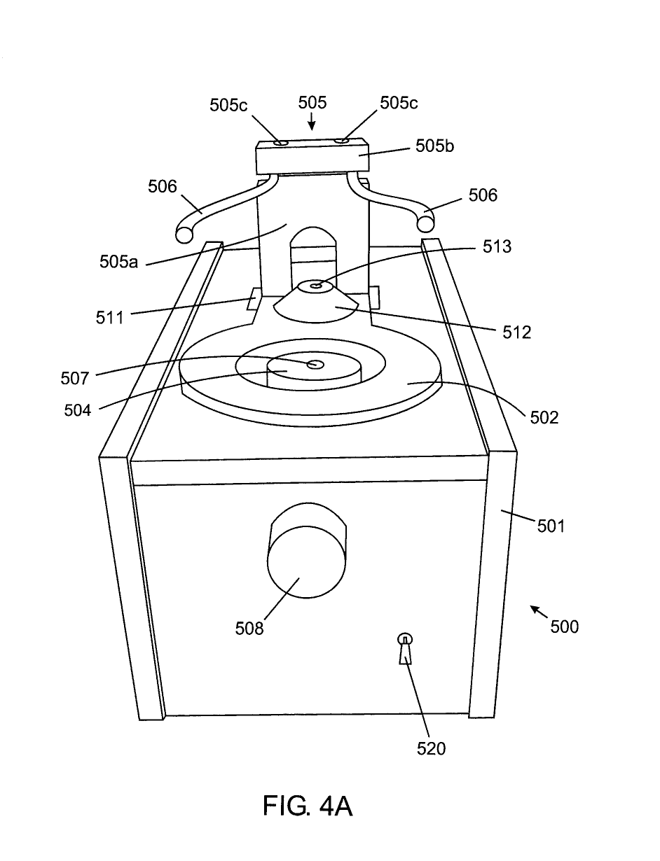

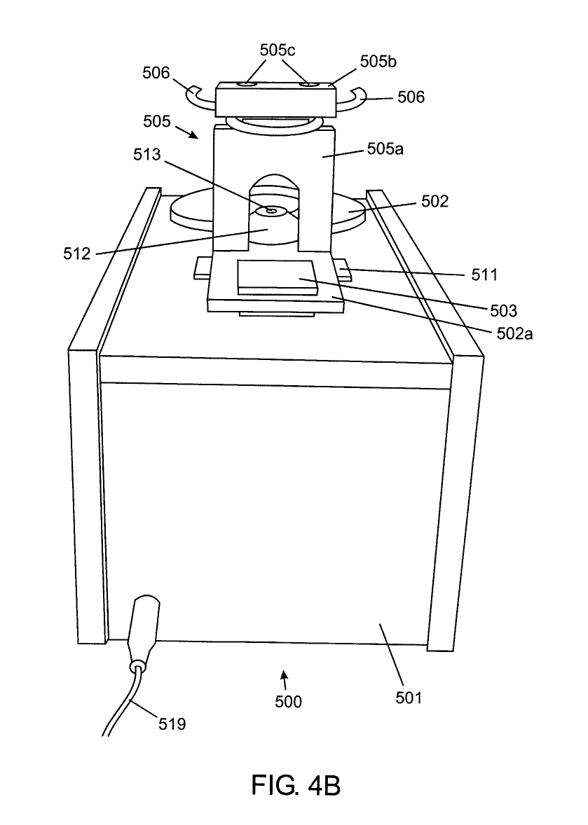

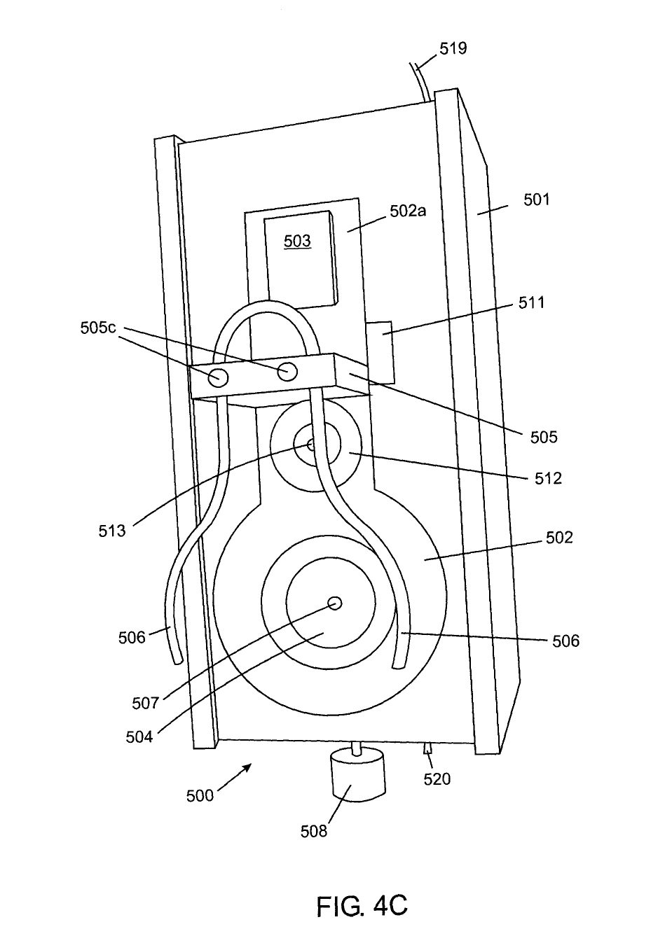

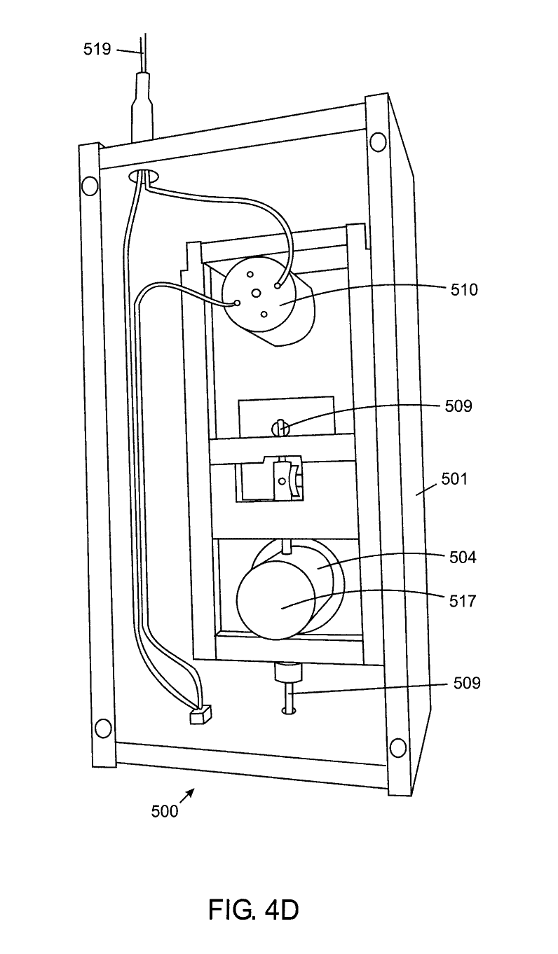

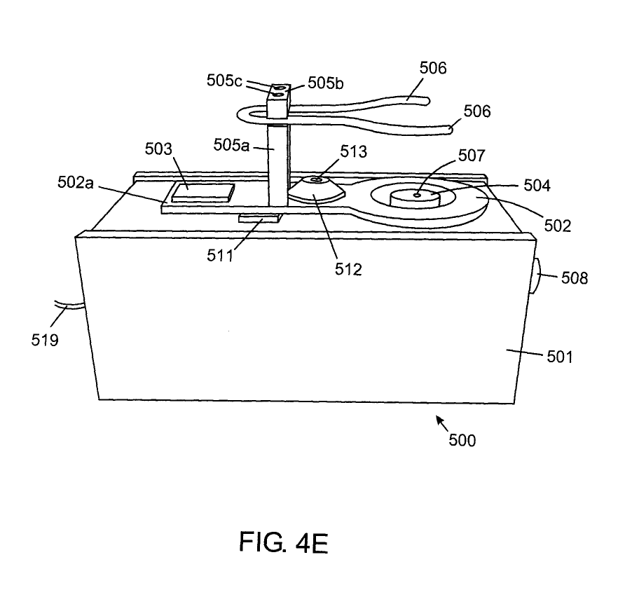

Referring to FIGS. 4A-4E, a second embodiment of the magnetic particle concentrator is now described. In this embodiment, magnetic particle concentrator 500 comprises a support structure for supporting the flask, a magnet positioned below the flask for concentrating the immunomagnetic beads into the center of the bottom of the flask, and means for vibrating the flask.

The support structure comprises base 502 upon which the flask rests, housing 501 for housing components of the concentrator and arm support 505 for supporting arms 506 which help hold the flask in place. The arms are unitized in a single elongated generally U-shaped element with curved ends to accommodate the contours of the flask. Arm support 505 comprises lower block 505a and upper block 505b. Supporting arms 506 are clamped in place between the lower block and upper block with the upper block bolted to the lower block by bolts 505c.

Magnet 507 and mirror 517 are mounted on opposite faces of support cylinder 504 which is mounted on magnet support shaft 509. The magnet support shaft is rotatable by actuation (e.g. rotation) of handle 508 connected to the shaft. Magnet 507 is initially positioned just below the center of the bottom of the flask. Once the magnetic particles have been concentrated, rotating the handle through 180-degrees moves the magnet down and away from the flask and raises the mirror to the position previously occupied by the magnet. The mirror assists in retrieving the magnetic particles by aiding visualization of the concentrated particles from below the flask. Rotating the handle through another 180-degrees returns the magnet to its position just below the flask. Rotation can be clockwise or counterclockwise.

The means for vibrating the flask comprises base 502 on which the flask rests. The base is a unitized plate having a generally annular portion on which the flask rests and generally rectangular portion 502a which extends from the generally annular portion under arm support 505 toward the rear of the apparatus. Proximal the end of generally rectangular portion 502a, a drive shaft of electric motor 510 (7.4 V, 0.6 A) is engaged with vibration block 503 which sits tightly within an aperture in generally rectangular portion 502a of the base. When the motor is switched on, the motor causes the vibration block to vibrate and vibrations from the vibration block are transmitted through the generally rectangular portion of the base which is in contact with the vibration block. Vibrations from the generally rectangular portion are transmitted through the base to the generally annular portion which in turns vibrates the flask resting on the generally annular portion. Vibration isolator 511 reduces transmission of vibrations to housing 501 when the motor is on. Vibrations reaching the generally annular portion of base 502 from generally rectangular portion 502a can be controlled by vibration controller 512. The vibration controller comprises a frustoconical element connected to the housing through the base by bolt 513. Tightening bolt 513 engages the frustoconical element more tightly against the base thereby restricting motion of base 502 thereby reducing transmission of vibrations from the generally rectangular portion to the generally annular portion. Loosening bolt 513 permits greater freedom of motion for base 502 thereby increasing transmission of vibrations from the generally rectangular portion to the generally annular portion resulting in more vigorous vibration of the flask. The motor is switched on and off by actuation of switch 520, and electricity is supplied to the motor from power cord 519, which may be connected to a step-down transformer.

To concentrate immunomagnetic beads at the center of the bottom of a flask resting on the generally annular portion of base 502, the flask is placed in the concentrator between arms 506 such that the arms are in contact with the flask, and magnet 507 is raised to its position just below the flask. Motor 510 is switched on and the motor rotates the drive shaft which vibrates vibration block 503 which in turn vibrates generally rectangular portion 502a of base 502. Vibration of base 502 is transmitted to the flask and thence to the flask's contents. Magnet 507 attracts the beads toward the center of the bottom of the flask, and vibration of the flask causes the beads to be slightly raised off the bottom of the flask thereby assisting in movement of the beads by eliminating friction between the beads and the bottom of the flask. Once the beads have been concentrated in the center of the bottom of the flask, magnet 507 may be rotated away from the bottom of the flask to be replaced by mirror 517, which assists in visualizing the location of the beads for retrieval.

### Second and Third Embodiments of Magnetic Particle Collector

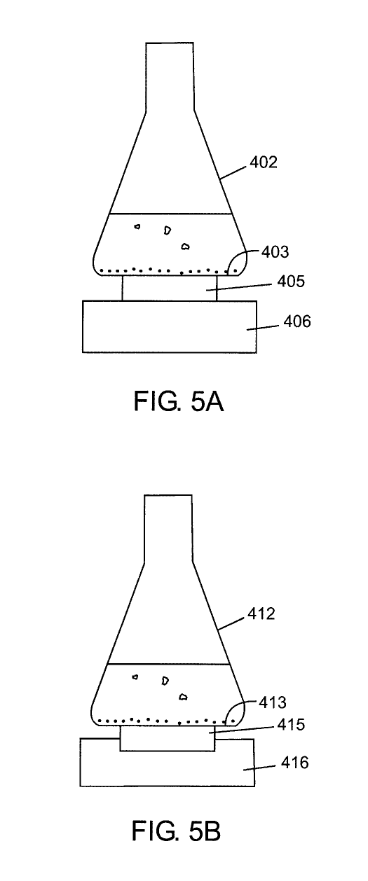

Referring to FIGS. 5A and 5B, second and third embodiments of magnetic particle collectors are now described.

A second embodiment of a magnetic particle collector as depicted in FIG. 5A comprises a powerful neodymium-iron-boron magnet 405 of a size covering at least half of the surface area of bottom 403 of Erlenmeyer flask 402. The magnet is attached to and sits on the surface of backplate 406.

A third embodiment of a magnetic particle collector as depicted in FIG. 5B comprises a powerful samarium-cobalt magnet 415 of a size covering at least half of the surface area of bottom 413 of Erlenmeyer flask 412. The magnet is recessed in the surface of backplate 416.

In both the second and third embodiments of the magnetic particle collector, the backplate has the following features. It comprises a material of high magnetic permeability and susceptibility, for example mild steel or transformer iron. It is larger than the magnet and is approximately the same diameter as the flask. It has a thickness of at least one-fifth of it diameter. The backplate is in good contact with the magnet and is more or less symmetrically disposed around the magnet. The backplate modifies the magnetic field around the magnet to increase the magnet's strength in an upwards direction toward the flask. The backplate increases by about 3-fold the magnetic field strength experience by contents of the flask.

Any enclosures and/or protective covers may protect both the magnet and the backplate.

### Third Embodiment of Magnetic Particle Concentrator

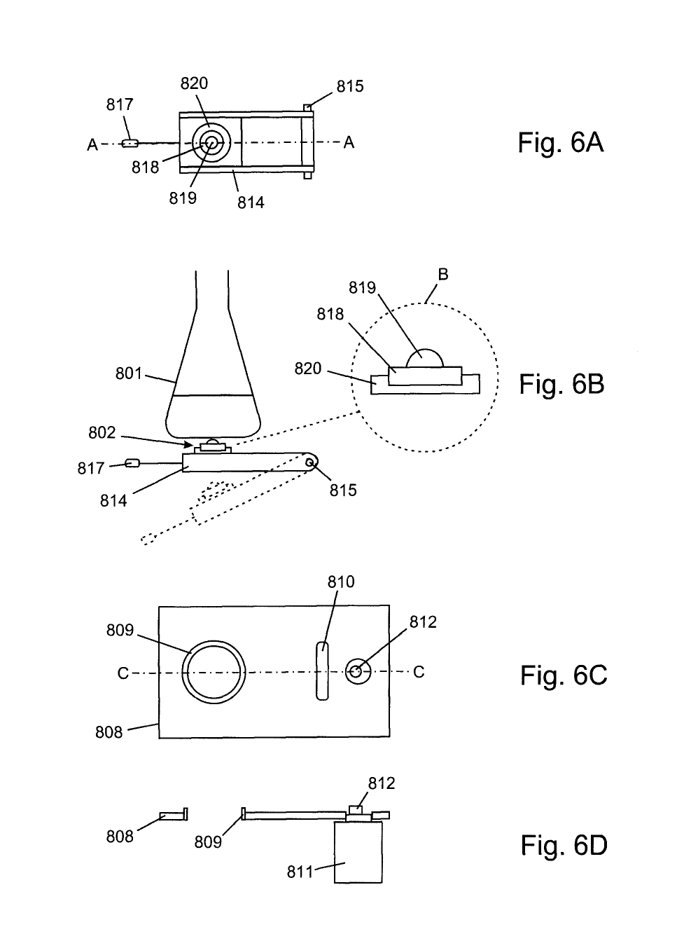

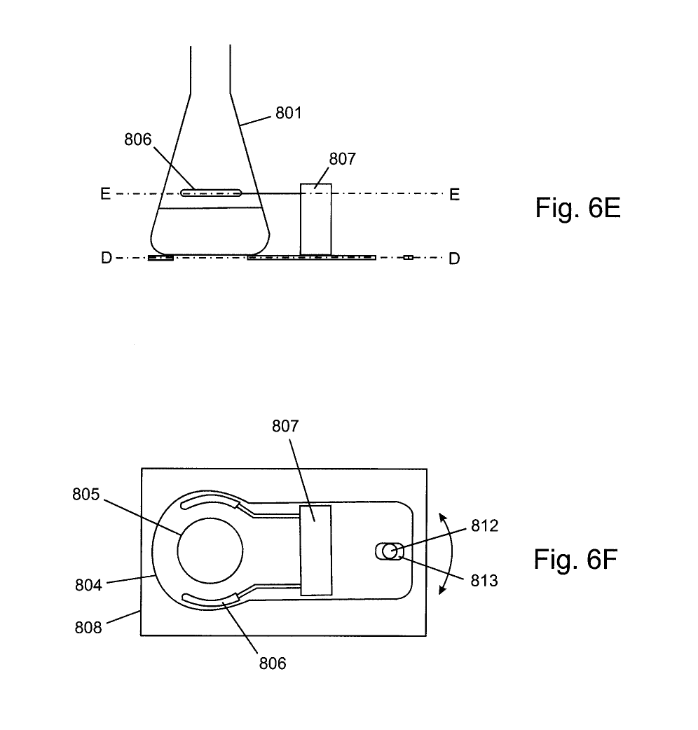

Referring to FIGS. 6A-6F, a third embodiment of the magnetic particle concentrator is now described.

Referring specifically to FIGS. 6A and 6B, a tilting frame 814, illustrated in plan in FIG. 6A and in section in FIG. 6B along line A-A in FIG. 6A, carries magnet assembly generally denoted at 802. Tilting frame pivots on pivot 815 allowing magnet assembly 802 to be raised to a raised position (solid lines in FIG. 6B) and lowered to a lowered position (dotted lines in FIG. 6B) as required so that it is either in contact with the centre of the bottom of flask 801 or far enough away that the magnetic field has a negligible effect on magnetic particles in the flask. Actuation of handle 817 raises and lowers tilting frame 814. A suitable detent means is be used to keep the tilting frame and therefore the magnet assembly in either the raised or lowered positions.

Magnet assembly 802 produces a strong magnetic field in a direction more or less parallel with the bottom of flask 801 extending to the perimeter of the bottom of the flask. The magnetic field is stronger at its centre so that all magnetic particles in the flask, including those close to or touching the bottom of the flask, experience an attraction toward the centre of the bottom of the flask, while those close to the centre experience an accentuated attraction toward the centre. As illustrated in blow-out B in FIG. 6B, the magnet assembly comprises disk or cylindrical magnet 818 carrying at its centre hemispherical magnet 819 with backplate 820 of high permeability and susceptibility to increase magnetic strength in an upward direction in a similar manner to that of the backplate illustrated in FIG. 5. Both magnets are of either neodymium-iron-boron or samarium-cobalt alloys. Other conformations could be used, for example, a single conical magnet in place of magnets 818 and 819.

A step in the process of the present invention is to draw magnetic particles in flask 801 towards the centre of the flask and to concentrate them into an easily removable pellet. Concentration is assisted by vibrating the flask about its vertical axis and above and close to the magnets.

Referring specifically to FIGS. 6C and 6D, chassis 808 illustrated in plan and section along line C-C, carries motor 811 of suitable speed. On the shaft of motor 811 and projecting through chassis 808 is an eccentric 812 of suitable eccentricity. Chassis 808 also carries flanged circular bushing 809 and low-friction surface 810 such that vibrating plate 804 can be vibrated about a vertical axis centered on bushing 809. Means, such as an upper low-friction surface (not shown) that presses down on plate 804 may be used to hold vibrating plate 804 securely close to chassis 808 while it is vibrating.

Referring specifically to FIGS. 6C to 6F, vibrating plate 804, illustrated in plan view in FIG. 6F showing planes along D-D and E-E from FIG. 6E, has circular cutout 805 such that vibrating plate 804 can rotate smoothly on bushing 809. Slot 813 slidingly accepts eccentric 812 such that the rotating eccentric 812 smoothly transmits an oscillatory motion to vibrating plate 804 in plane D-D. Stanchion 807 on vibrating plate 804 carries springy clamp arms 806 at a suitable height and of a shape to grip the periphery of flask 801 to hold the flask firmly with its axis centered above the centre of bushing 809. Clamp arms 806 may be covered in resilient high-friction material such as a rubber where they contact the flask so that the oscillation of vibrating plate 804 is transmitted efficiently to the flask causing it to oscillate about its axis.

To use the apparatus, flask 801 is inserted into clamp arms 806. Magnet assembly 802 is brought up into contact with the bottom of the flask and motor 811 is switched on. Rapid oscillation of the flask about its axis overcomes forces that cause the magnetic particles to stick to the bottom of the flask thereby allowing the magnetic particles to migrate under the influence of the magnetic field towards the centre of the flask where the magnetic field is most intense. When the particles come close to the centre of the flask the very localized intense central magnetic field from magnet assembly 802 causes the particles to coalesce into a small pellet or button. At this point, motor 811 is stopped, and when the flask has come to rest the button of magnetic particles can be removed.

### Second Embodiment of Magnetic Particle Pipette

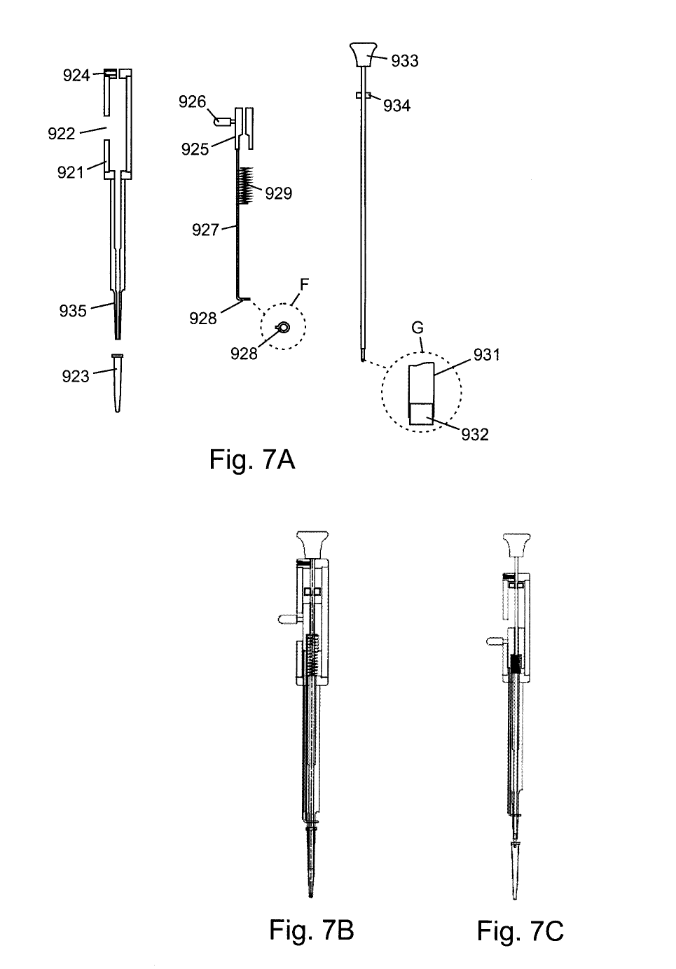

FIGS. 7A-7C illustrate a second embodiment of a magnetic particle pipette in which FIG. 7A depicts various components of the pipette disassembled into three parts for clarity, FIG. 7B depicts an assembled pipette in a rubber tip-attaching configuration and FIG. 7C depicts the assembled pipette in a rubber-tip detaching configuration.

Tubular pipette body 921, conveniently of circular cross-section and of any suitable material (e.g. metal or rigid plastic), has slot 922 in a higher region of the tubular pipette body such that first knob 926 attached to slider 925 can move freely up and down. A smaller diameter section 935 in a lower region of the tubular pipette body has a diameter such that it can removably grip rubber tip 923. Rubber tip 923, fabricated of inert elastic material, is tapered and is closed at its narrow end. The rubber tip is conveniently a commercially available product sold under the trade name PickPen™ Tip by BIOCONTROL System, Inc. WA, USA. Friction plug 924 placed conveniently at the top of tubular pipette body 921 is useful to slidably hold magnet shaft 930 at whatever position a user desires. Slider 925 carrying first knob 926 and push-off wire 927 fits slidably inside tubular pipette body 921 and is conveniently maintained in a raised position by spring 929, unless the user actuates it by pressing downwards on first knob 926. Push-off wire 927 is looped (see blow-out F) at its bottom end 928 to loosely encircle the narrowed end of tubular pipette body 921 just above the top of rubber tip 923 when slider 925 is in its raised position.

Magnet shaft 930 is of a suitable diameter and stiffness to slide up and down in tubular pipette body 921. At its lower tip, magnet shaft 930 carries a small magnet 932, preferably of neodymium-iron-boron or samarium-cobalt alloy, seated within a surrounding seat 931 of high magnetic permeability and susceptibility material to increase magnetic field strength in the downward direction (see blow-out G). Magnet shaft 930 also has second knob 933 by which the user can raise and lower magnetic shaft 930, and stop 934 to prevent the user from raising the magnetic shaft too far. Magnetic shaft 930 is prevented from falling from the raised position by friction plug 924 as needed.

To use the magnetic particle pipette, the user presses tubular pipette body 921 into rubber tip 923 so that rubber tip 923 remains attached to the body by friction, and then presses down second knob 933 to push magnet 932 into the tip of rubber tip 923 as illustrated FIG. 7B. When the user then dips the pipette into a button of magnetic particles in the centre of the bottom of a flask, the magnetic particles are strongly attracted to magnet 932 and adhere to the outside of the tip of rubber tip 923 allowing them to be pulled out of the flask and introduced into a receiving vessel for succeeding stages of purification. The user then raises second knob 933 by thumb or finger to draw magnet 932 upwards thereby reducing the magnetic field holding the button of magnetic particles against rubber tip 923. This allows the magnetic particles to fall off into the receiving vessel. Finally, as illustrated in FIG. 7C, the user can move first knob 926 downwards causing loop 928 of push-off wire 927 to push the now contaminated rubber tip 923 from the pipette.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. Apparatus for concentrating magnetic particles into a localized region on a bottom of a container, the apparatus comprising: a support structure for supporting the container; a magnet positioned below the container when the container is supported on the support structure, the magnet having a magnetic field localizable at the region on the bottom of the container to concentrate the magnetic particles into the region, the magnetic field removable from the region to permit retrieval of the magnetic particles without interference from the magnet; and, means for vibrating the container to assist in movement of the magnetic particles to the localized region.

2. The apparatus of claim 1, wherein the magnet is a permanent magnet.

3. The apparatus of claim 1, wherein the magnet is an electromagnet.

4. The apparatus of claim 2, wherein the magnet has a surface smaller than the bottom of the container, the surface of the magnet determining size of the localized region.

5. The apparatus of claim 4, wherein the magnet is positioned to be under a center of the bottom of the container.

6. The apparatus of claim 1, wherein the magnet is physically movable away from the localized region.

7. The apparatus of claim 6, wherein the magnet is physically movable up and/or down by action of a lever or a rotatable shaft.

8. The apparatus of claim 1, wherein the means for vibrating the container comprises one or more vibrating arms or a vibrating base.

9. System for recovering magnetic particles from a mixture in a container, the system comprising: a magnetic particle collector having a first magnet for attracting the magnetic particles to a bottom of the container; a magnetic particle concentrator having a second magnet for concentrating the magnetic particles collected on the bottom into a localized region on the bottom, the concentrator having means for vibrating the container to assist in movement of the magnetic particles to the localized region; and, a magnetic particle pipette having a third magnet for retrieving the magnetic particles concentrated in the localized region.

10. The system of claim 9, wherein the container has a flat bottom.

11. The system of claim 9, wherein the container is an Erlenmeyer flask.

12. The system of claim 9, wherein the first magnet is a permanent block magnet.

13. The system of claim 9, wherein the first magnet is an electromagnet.

14. The system of claim 10, wherein the second magnet is a permanent magnet having a surface smaller than the bottom of the container, the surface of the second magnet determining size of the localized region.

15. The system of claim 10, wherein the second magnet is an electromagnet having a surface smaller than the bottom of the container, the surface of the second magnet determining size of the localized region.

16. The system of claim 10, wherein the means for vibrating the container comprises one or more vibrating arms or a vibrating base.

17. The system of claim 10, wherein the second magnet is physically movable up and/or down under the localized region of the container.

18. The system of claim 9, wherein the third magnet is an electromagnet.

19. Process for selectively isolating microorganisms from a sample, the process comprising: providing a container containing a medium for selectively enriching the microorganisms; adding the sample containing the microorganisms to the medium; adding to the medium magnetic particles adapted to bind the microorganisms of interest; magnetically collecting the particles with bound microorganisms at a bottom of the container; aided by vibrating the container, magnetically concentrating the particles with bound microorganisms at a localized region on the bottom of the container; and retrieving the particles from the localized region with a magnetically assisted pipette.

20. The process of claim 19, wherein the microorganisms are Shigella spp.

21. The process of claim 19, wherein the container is an Erlenmeyer flask.

22. The process of claim 19, wherein the medium comprises water, tryptone, potassium phosphate dibasic, potassium phosphate tribasic, sodium chloride, glucose, polyethyleneglycol sorbitan monooleate, and Crystal Violet.

23. The process of claim 19, wherein the magnetic particles comprise immunomagnetic beads having antibodies specific to the microorganisms.
